(19) United States
(12) Patent Application Publication
(10) Pub. No.: US 2020/0008910 A1
(43) Pub. Date: Jan. 9, 2020

(54) Multi-Layer Molded Part

(71) Applicant: Vita Zahnfabrik H. Rauter GmbH & Co. KG, Bad Sackingen (DE)

(72) Inventors: Enno Bojemueller, Bad Sackingen (DE); Michael Gorbar, Dubendorf (CH); Dirk Penner, Zurich (CH); Michael Tholey, Bad Sackingen (DE)

(21) Appl. No.: 16/504,899

(22) Filed: Jul. 8, 2019

(30) Foreign Application Priority Data
Jul. 9, 2018 (EP) 18182507.6

(51) Int. Cl. A61C 13/00; A61C 13/08; A61C 5/77; C04B 35/18; C04B 35/486; C04B 35/626; C04B 35/634; C04B 35/636; B32B 18/00

(52) U.S. Cl. CPC A61C 13/0004; A61C 13/081; A61C 13/0006; A61C 5/77; C04B 35/18; A61C 13/083; C04B 35/6261; C04B 35/6342; C04B 35/636; B32B 18/00; C04B 35/486

(57) ABSTRACT

A molded part having a multitude of ceramic layers provided on top of one another, and a process for the preparation thereof are described. Further, the use of a multi-layer molded part for preparing a dental restoration is described.

### CROSS REFERENCE TO RELATED APPLICATIONS

This application claims priority to and the benefit of European Patent Application No. 18182507.6 filed in the European Patent Office on Jul. 9, 2018, which is incorporated herein in its entirety by reference thereto.

### BACKGROUND OF THE INVENTION

The present invention relates to a molded part having a multitude of ceramic layers provided on top of one another, and a process for the preparation thereof. In addition, the invention relates to the use of a multi-layer molded part for preparing a dental restoration.

Ceramic materials are characterized by a wide variety of properties that cannot be realized with other materials, such as hardness, durability, abrasion resistance, corrosion resistance, and thermal and electrical insulation.

Because of their different properties, ceramic materials find abroad application, from the production of everyday items, such as dishes or tiles, to the use in electronics, information technology, or as biomedical prostheses. Especially because of the progressing development of CAD/CAM technology, directly processable ceramics or ceramics that can be dense-sintered after processing have increasingly moved into the focus and tend to capture the field of manually prepared dental restorations.

In contrast to metals, ceramics mostly have brittleness as material properties, which can be optimized by suitable stabilization mechanisms or material compositions. Yttria-stabilized zirconium dioxide or polymer-infiltrated ceramics (VITA ENAMIC®) may serve as appropriate examples. Particular attention in the production of ceramic dental restoration materials is drawn to the sintering process, which is performed for attaining the required stability. The sinter shrinkage, which occurs during sintering, must be calculated exactly to be able to adjust the desired material properties in a reproducible way. In order to ensure a corresponding shaping, ceramics are mostly processed into free-flowing pressed granules using pressing additives, which granules are pressed into molded parts, such as blocks or disks, on automatic presses. For larger molded parts, isostatic after compaction may be employed in order to minimize structural inhomogeneities. In the case of yttrium-stabilized zirconium dioxide, debindering takes place followed by sintering to a porous stage, in which the material can be processed by a CAD/CAM method. The dental restoration is ground to a larger size and subsequently dense-sintered in order to attain the required fit and also the desired material properties. The color of the restoration is also achieved by the step of dense-sintering. In the case of an opaque zirconium dioxide with, for example, 3 mole percent tetragonal stabilization by yttria, the thus obtained restoration is subsequently veneered manually with a veneering ceramic. In the case of, for example, an increasingly cubic 6 mole percent stabilization with yttria, the sintered final product may already have the desired tooth color after dense-sintering because of its higher translucency, and need not be veneered subsequently.

In the case of polymer-infiltrated ceramics (hybrid ceramics, Vita ENAMIC), the ceramic component is also provided with press additives, debindered and sintered to the desired porosity. In the following step, the continuous porous network is infiltrated with a monomer mixture admixed with a polymerization initiator, and polymerized so that no cavities remain in the structure. This hybrid material now has the final tooth color and can be processed directly by a CAD/CAM method to a dental restoration.

A particular difficulty in the preparation of dental restorations resides in the fact that natural teeth are not single-color, but have a complex color gradient, which must be taken into account in order to achieve an aesthetically satisfactory result. Thus, different regions of the same tooth may differ in color and transparency. In general, the color and transparency gradient extends from transparent in the occlusal region to yellowish-opaque in the cervical region. In order to achieve a desired color design, dental restorations may be veneered afterwards. However, such a veneering usually involves a lot of manual effort and requires a dental-engineering education in the veneering materials employed. In addition, the mechanical properties in the tooth also differ, the more elastic dentin having a clearly lower bending strength as compared to the clearly more brittle enamel, which is used to comminute the food. However, the two components together result in a highly loadable tooth, whose more flexible interior part and the root, the dentin, cushion the forces applied to the enamel. These properties must also be reflected by the dental restoration. Because of such demands and before the background of increasing rationalization, the use of molded parts for the preparation of dental restorations is preferred, meeting the aesthetic and mechanical requirements even without a veneer, and having a color design that comes as close as possible to the natural teeth in terms of color and transparency as well as color gradient.

EP 1 900 341 discloses molded parts of several layers of different colors with a specific sequence of layers in order that the color transition between the layers is not visible. The molded parts are prepared by dry pressing layered glass-ceramic powders having different colors, debindering and sintering. Dental restorations can be prepared from such a molded part by means of CAD/CAM methods.

WO 2008/144388 provides multi-layer ceramic bodies and systems, which are prepared by means of strip casting and reaction bonding technologies and have an improved structural reliability as compared to ceramic compositions prepared by conventional methods. The ceramic bodies and systems described may be used for any kind of biomedical prostheses and are particularly useful as dental restorations.

WO 2008/083358 describes a dental blank having at least one inner zone or layer of a first color and an outer zone or layer of a second color, in which the inner and outer zones are arranged concentrically. The inner zone may be entirely enclosed by the outer zone, so that only the outer zone is visible on all surfaces of the blank, but not the inner zone. Alternatively, the inner and outer zones may extend to the same surface of the blank, so that only the outer zone covers the remaining surfaces of the blank. In addition, the dental blank may have an intermediate zone between the inner and outer zones, in which the intermediate zone is entirely enclosed by the outer zone, and/or the intermediate zone encloses the inner zone entirely.

The molded parts described in the prior art have the disadvantage that a partially visible color transition occurs between two layers of different colors, and in translucent materials, there is further a problem in that a gray color, which can be recognized as a dark or bright stroke in the dental restoration, occurs between the layers of different colors. In addition, only a limited number of layers can be provided on top of one another with conventional pressing methods, because the number of channels available in the software for moving a filling device necessary for production is limited. In addition, the limited space in the press space does not allow for any number of different colored press granules, so that a natural color gradient is not accessible in this way in addition to the limitation to the granules and layer thickness.

Therefore, there is still a need for ceramic molded parts having properties that can be modulated, especially in the field of dental restorations, in order to reflect the natural color gradient and the natural mechanical properties of a tooth, so that the dental restoration fits itself into the existing tooth structure in accordance with aesthetic requirements.

### BRIEF SUMMARY OF THE INVENTION

Objects and advantages of the invention will be set forth in part in the following description, or may be obvious from the description, or may be learned through practice of the invention.

In one particular embodiment of the present invention, a molded part having a multitude of ceramic layers provided on top of one another is described. The present invention further contempaltes a process for the preparation of a molded part as well as a dental restoration formed from the molded part.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE FIGURES

A full and enabling disclosure of the present invention to one skilled in the art, including the best mode thereof, is set forth more particularly in the remainder of the specification, including reference to the accompanying figures, in which:

FIG. 1 shows a scanning electron micrograph of a molded part according to the invention, in which the crack-free transition from 4.5 μm through 1.4 μm to 2.6 μm thick layers in a dense-sintered state can be clearly seen.

FIG. 2 shows a scanning electron micrograph of another molded part according to the invention, in which the crack-free transition from 4.5 μm to 2.6 μm thick layers in a dense-sintered state can be clearly seen.

FIG. 3 shows a stereomicroscopic image of a molded part according to the invention, which consists of alternate layers of ZrO₂ and feldspar ceramics and has been prepared by the process according to the invention. The bonding between the two different materials can be clearly seen here.

FIG. 4 shows a scanning electron/BSD micrograph of a molded part according to the invention, wherein the highlighted region shows the transition from porous-sintered ZrO₂ to feldspar ceramics with a crack-free bonding without porosity.

FIG. 5 shows a scanning electron micrograph of a molded part according to the invention made of porous-sintered feldspar ceramics in different grain sizes with an almost dense structure (top) and two different porosities resulting from different grain sizes (middle and bottom).

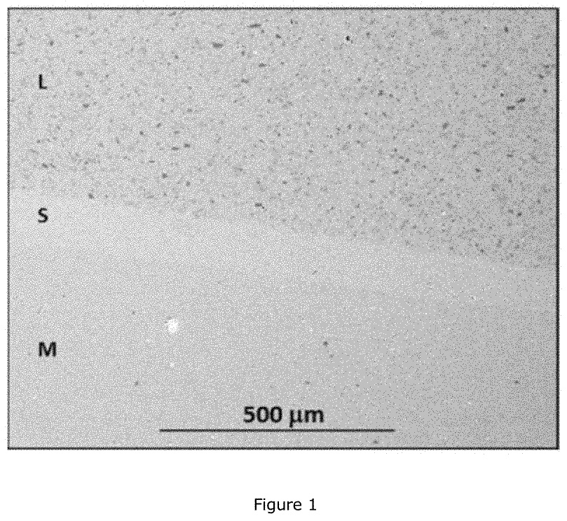

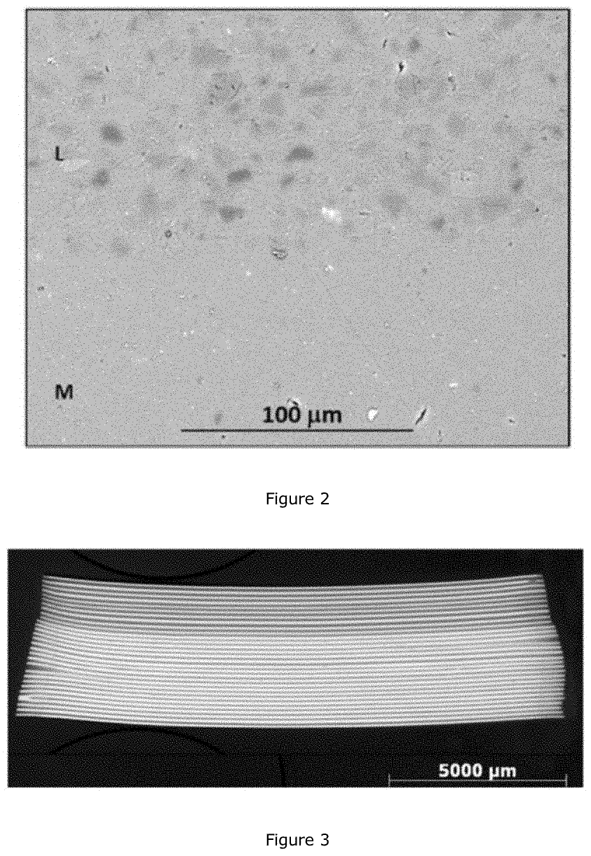

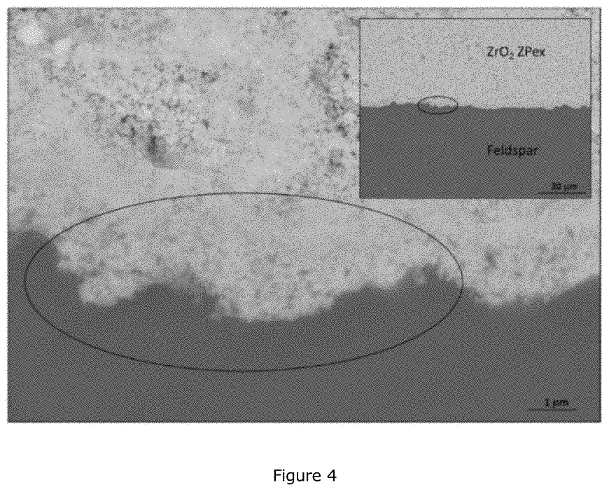

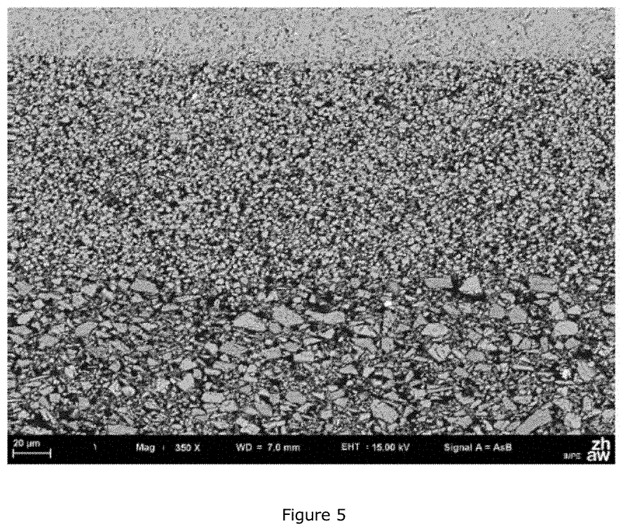

Repeat use of reference characters in the present specification and drawings is intended to represent the same or analogous features or elements of the present invention.

### DETAILED DESCRIPTION OF REPRESENTATIVE EMBODIMENTS

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

One object of the present invention is to provide a ceramic molded part whose properties can be adapted to the individual optical and functional needs of the respective application.

This object is achieved by a molded part having a plurality of ceramic layers provided on top of one another. Surprisingly, it has been found that both the optical properties of the molded part, such as the color gradient, and mechanical and structural properties, such as the sintering performance, can be adapted by a layer structure.

Therefore, the present invention firstly relates to a molded part having a plurality of ceramic layers provided on top of one another, wherein at least 21 layers are provided on top of one another.

“Ceramic layers” within the meaning of the present invention means layers or stratums in the molded part that comprise at least one ceramic material. In order to be able to adjust the properties of the molded part as precisely as possible, it has been found advantageous if the number of the ceramic layers of the molded part is more than 21, preferably more than 25, more preferably more than 50, and especially more than 100.

It is further advantageous, especially with respect to the optical properties, if the layer thickness of the individual layers does not exceed a particular thickness. In this way, a continuous change of the properties of the different layers, such as a color gradient, can be achieved, so that the transitions between the individual layers can no longer be seen by the naked eye. Therefore, in a preferred embodiment of the molded part according to the invention, the layers have a thickness of from 1 μm to 120 μm, especially from 1 to 99 μm, preferably from 10 μm to 80 μm, more preferably from 30 μm to 60 μm.

The properties of the molded part can be adjusted by the ceramic layers having different properties, such as color, density, porosity, absorption behavior, and X-ray opacity, or by using different materials. Therefore, an embodiment is preferred in which the layers of the molded part according to the invention that are provided on top of one another respectively comprise the same ceramic material, or at least one of the ceramic layers provided on top of one another comprises a ceramic material different from those of the other ceramic layers.

Preferably, the ceramic layers of the molded part according to the invention comprise a ceramic material selected from the group consisting of feldspar ceramics, metal oxide ceramics, non-oxidic ceramics, polymer-infiltrated hybrid ceramics, glass ceramics, and combinations thereof. In a particularly preferred embodiment, the ceramic material is selected from the group consisting of lithium silicate ceramics, aluminum oxide ceramics, zirconium oxide ceramics, stabilized zirconium oxide ceramics, leucite ceramics, cordierite ceramics, glass ceramics reinforced by crystallites, fiber-reinforced ceramics, and combinations thereof. These embodiments can be employed in a densely sintered form or also in a porously sintered form with a subsequent infiltration step.

The molded parts according to the invention allow for the preparation of dental restorations with a natural appearance, which integrate themselves in the environment of natural teeth without optical differences. In particular, it has been surprisingly found that the color transition in the restoration can be realized continuously by using different materials. Therefore, one embodiment in which the molded part according to the invention is constituted of alternating layers of zirconium dioxide and feldspar is particularly preferred.

Molded parts that are used, in particular, for preparing dental restorations must have a high mechanical loading capacity, which withstands the forces that are acting on the restoration during the chewing process. In order to achieve a corresponding loading capacity, it has been found advantageous if the molded part comprises dense-sintered ceramic particles. Therefore, an embodiment of the molded part according to the invention is preferred in which at least one of the ceramic layers has dense-sintered ceramic particles.

Further preferred is an embodiment of the molded part according to the invention in which the ceramic layers have different densities. Particularly preferred is a molded part in which the ceramic layers of different densities form a density gradient. “Density gradient” within the meaning of the present application means a continuous change of the density throughout the solid or over part of the molded part, wherein said density gradient is formed by providing several layers of different densities on top of one another.

In order to adjust the mechanical and optical properties of the molded part according to the invention, it may be of advantage if the molded part comprises other materials incorporated into the ceramic structure, in addition to the ceramic material(s). This is of advantage, in particular, for applications in dental restoration, because an equilibrium between the required mechanical loading capacity and a good processability is achieved in this way. Also in other fields, such as electronics, such a possibility of combining different materials may be advantageous. Suitable methods for preparing such materials are described, for example, in the industrial right documents WO 2002/076907, EP 1 238 956, and WO 2010/029515, which are incorporated herein by reference.

Accordingly, an embodiment of the molded part according to the invention is preferred in which at least one of the ceramic layers has pores formed by porous-sintered ceramic particles. In this way, not only the density of the molded part can be varied. The pores also enable the incorporation of other materials, such as polymers, into the ceramic structure.

In a preferred embodiment, the pores of the ceramic layers comprising porous-sintered ceramic particles are completely or at least partially filled with another material. More preferably, the other material is selected from the group consisting of plastic, ceramic, glass and metal. In the case where the other material is a ceramic, this ceramic preferably differs from the ceramic material of the layers of the molded part according to the invention.

Preferably, the pores have a pore size of from 10 nm to 100 μm, but may also deviate therefrom in particular embodiments. Because of the combination of layers with different porosities, different pore sizes occur in the individual layers. The pore sizes are average pore sizes, which normally have a size distribution.

Further preferred is an embodiment of the molded part according to the invention in which a plurality of ceramic layers with porous-sintered ceramic particles are provided on top of one another respectively in direct vicinity to form a porous system extending through the different layers. Preferably, at least 70% of the ceramic layers have porous-sintered ceramic particles, preferably at least 80%, and more preferably from 85% to 99%.

In a preferred embodiment, the molded part according to the invention has a porosity of 5-99%, preferably 7-95%, more preferably 10-90%. In an also preferred embodiment, at least one of the ceramic layers has a porosity of 5-99%, preferably 7-95%, more preferably 10-90%. In a particularly preferred embodiment, at least two of the ceramic layers have different porosities.

The physical and chemical properties of the molded part according to the invention can be influenced by adding aggregates to the ceramic material, wherein the kind and quantity of the aggregates in the different ceramic layers may vary. Therefore, in a preferred embodiment, at least one ceramic layer has at least one aggregate for adjusting physical properties, such as the color, translucency, opacity, X-ray opacity, opalescent effects, fluorescence, bending strength and/or modulus of elasticity, wherein this list it to be understood as exemplary.

In a preferred embodiment, the molded part according to the invention or at least one of its layers contain further aggregates, especially those selected from the group consisting of low- or high-melting frits or chemicals such as sodium carbonate or borax, and additives containing leucite. It has been found that the sintering properties can be influenced by adding suitable additives, such as low- or high-melting frits or chemicals such as sodium carbonate or fluxes such as borax. Leucite-containing additives can be used to influence the thermal expansion coefficient of the ceramic.

Preferably, the aggregate is selected from the group consisting of dyeing substances, nucleating agents, fluxes, refining agents, ceramic fibers, nanomaterials, stabilizers, and mixtures thereof. In addition to the need for the molded parts themselves, there is also a need for a process that enables the preparation of the molded parts described.

Therefore, the present invention further relates to a process for preparing the molded part according to the invention, comprising the following steps:
a) providing a slip comprising a ceramic material;
b) applying the slip to a support material in a desired layer thickness;
c) drying the layer from step b) to obtain a single ceramic layer;
d) repeating steps a) to c) until the desired number of ceramic layers is achieved;
e) arranging the individual layers from step d) to obtain a stack arrangement; and
f) pressing the stack arrangement from step e) to obtain a molded part.

In a preferred embodiment, the molded part is sintered. In this way, a sufficient stability of the ceramic structure can be achieved. In an alternatively preferred embodiment, the non-sintered stack arrangements are debindered or washed out or freed from organic material by dissolving it in suitable solvents. These graded structures with porosity or material gradients may also be subsequently infiltrated, then having a gradient of packing in the dense infiltrated structure.

In a preferred embodiment, the slip comprises ceramic powder, dispersing aids, and a liquid phase. This first slip may be prepared, for example, by milling and homogenizing the slip components. Preferably, a second slip is prepared by adding further components, such as rheological aids, liquefiers, defoamers, binders and/or cross-linking agents, with stirring, the second slip being applied to the support material. In a preferred embodiment, the liquid phase of the slip is water, inorganic or organic solvents, or mixtures thereof.

In a preferred embodiment, the applying of the slip to the support material is effected by blade coating, spraying or casting. Without being limited to particular methods for application, established methods may be used by way of example, such as spin coating, dip coating, blade coating, slot coating, or also spray coating. In this way, the desired layer thickness can be adjusted to an optimum value.

It should be possible to detach the dried layer from the support material non-destructively and without a residue. Preferably, the support material is selected from the group consisting of cellophane sheet, plastic plates or sheets made of different materials in different thicknesses, absorptive or non-absorptive, optionally coated, paper, metal foils or sheets, membranes for liquid removal, and similar support materials.

In a preferred embodiment, the molded part obtained may be processed after pressing, for example, by means of subtractive methods, such as CAD/CAM methods or laser milling. The processing may also be manual, for example, by means of manually operated milling tools. In an alternatively preferred embodiment, the pressed molded part can be processed by means of additive methods.

Depending on the material employed, it may be advantageous to sinter the molded part in advance before the processing in order to facilitate the processing. Therefore, an embodiment of the method according to the invention is preferred in which the molded part is sintered in advance after the pressing and before the processing, wherein the density attained is lower than the final density.

The desired strength of the molded part can be achieved by sintering, depending on the ceramic material employed. Accordingly, an embodiment is preferred in which the molded part is subjected to a sintering step. The sintering may be effected, for example, after the processing of the molded part. Alternatively, the sintering may be effected before the processing of the molded part.

The density to be attained depends on the desired use of the molded par. For example, the molded part can be sintered to more than 70% of its theoretical density, for example, more than 80% of its theoretical density, or more than 90% of its theoretical density. High densities associated with a high strength are desirable, in particular, when the molded part is used in the field of dental restorations. For other applications, it may be advantageous to sinter the molded part to, for example, 30% of its theoretical density.

The sintering of the processed molded part may cause it to shrink, and therefore, an embodiment is preferred in which this shrinkage factor is taken into account already in the processing, for example, the molding of a dental restoration, for example, by increasing the size of the dental restoration by this shrinkage factor.

A binder may be admixed with the slip for better processability. Therefore, in a preferred embodiment, the method according to the invention includes a step of debindering, in which the binder is removed, for example, by a thermal treatment, after the pressing of the molded part.

The process according to the invention allows for the preparation of a porous or partially porous molded part, in whose pores one or more further materials may be incorporated. For this purpose, the process according to the invention includes a step of silanization in a preferred embodiment. Surprisingly, it has been found that the silanization of the interior surface of the molded part achieves a better chemical bonding with the further material that is incorporated in the pores of the molded part. The silanization is preferably performed by contacting the molded part with a solution comprising a silicon-containing compound, preferably a silane. Said solution may be aqueous, partially aqueous or non-aqueous. Preferably, the molded part is subjected to a drying step after silanization.

The incorporation of the further material into the molded part is preferably effected after the silanization and subsequent drying of the molded part. Preferably, the incorporation is effected by contacting the molded part with said further material or materials. The further material may be, for example, a monomer or a monomer mixture, wherein said incorporation is optionally effected in the presence of a polymerization initiator. Preferably, one or more fillers may be admixed with the monomer or monomer mixture, for example, fillers for influencing the physical properties, such as the color, translucency, bending strength, X-ray opacity, opalescent effect, and/or fluorescence. For example, the filler may be dyeing substances, such as organic, inorganic and/or organic-inorganic mixed pigments. The incorporated monomer and/or the incorporated monomer mixture can then be polymerized in a subsequent step.

The process according to the invention provides the molded part with properties that are not accessible by conventional methods, for example, a homogeneous color gradient without transitions visible to the eye. Therefore, the present invention further relates to a multi-layer molded part obtained by the process according to the invention. As set forth above, the molded part according to the invention is particularly suitable for applications in the field of dental restorations. Therefore, the present invention further relates to the use of the molded part according to the invention for preparing a dental restoration. Surprisingly, it has been found that the use according to the invention can provide dental restorations that mimic the natural color gradient of the teeth surrounding them, without color transitions being visible to the human eye.

Preferably, said dental restoration is artificial teeth, inlays, onlays, bridges and/or crowns. It is further preferred that the preparation of the dental restoration is effected by a computer-aided method, especially by CAD/CAM methods in order to reach the required precision.

Accordingly, the present invention further relates to a dental restoration obtainable from the molded part according to the invention. Surprisingly, it has been found that the dental restorations according to the invention are characterized by a natural color gradient without visible color transitions.

The present invention further relates to a process for preparing a dental restoration using a molded part according to the invention, wherein the dental restoration is shaped from the molded part by ablating methods.

The present invention will be illustrated by means of the following Examples, which are not to be construed as limitations to the spirit of the invention.

The general procedure includes the milling of the starting material for preparing an agglomerate-free slip. This slip is adjusted with additives to form a castable slip for film casting. After applying the desired layer thickness by means of a doctor blade to a cellophane sheet and drying the thus produced ceramic sheets, the latter are trimmed, and stacked in the desired sequence, and pressed. The subsequent sintering step forms the ceramic block with the locally differing porosity or color to be achieved.

### EXAMPLE 1

Feldspar ceramic powder with a grain size D50=4.5 μm (L=large), colorless (A1a), and tooth color 2M2 (A1b):

Preparation of the starting slips A1a and A1b: grinding 100 g of ceramic powder with 0.6 g of anionic polyelectrolyte (dispersing aid) and 81.5 g of distilled water with 200 g ZrO₂ milling balls for 12 h, then sieving off the milling balls.

Preparing the slip for film casting: 86.6 g of starting slip A1a or A1b, 2.68 g of ethylene/vinyl acetate dispersion (temporary binder), 0.54 g of modified starch (binder/thickener), 5 g of ethanol/2% MEK (MEK=methyl ethyl ketone), and 0.1 g of 1-octanol are stirred with 500 rpm for 1 h, then mixed for 20 s at 2500 rpm in a speed mixer.

### EXAMPLE 2

Feldspar ceramic powder with a grain size D50=2.6 μm (M=medium), tooth color 3M2:

Preparation of the starting slip A2: grinding 100 g of ceramic powder with 0.6 g of anionic polyelectrolyte and 81.5 g of distilled water with 200 g ZrO₂ milling balls for 12 h, then sieving off the milling balls.

Preparing the slip for film casting: 86.6 g of starting slip A2, 2.68 g of ethylene/vinyl acetate dispersion, 0.54 g of modified starch, 5 g of ethanol/2% MEK (MEK=methyl ethyl ketone), and 0.1 g of 1-octanol are stirred with 500 rpm for 1 h, then mixed for 20 s at 2500 rpm in a speed mixer.

### EXAMPLE 3

Feldspar ceramic powder with a grain size D50=1.4 μm (S=small), tooth color 2M2:

Preparation of the starting slip A3: grinding 100 g of ceramic powder with 0.6 g of anionic polyelectrolyte and 81.5 g of distilled water with 200 g ZrO₂ milling balls for 12 h, then sieving off the milling balls.

Preparing the slip for film casting: 86.6 g of starting slip A3, 2.64 g of ethylene/vinyl acetate dispersion, 0.53 g of modified starch, 5 g of ethanol/2% MEK (MEK=methyl ethyl ketone), and 0.1 g of 1-octanol are stirred with 500 rpm for 1 h, then mixed for 20 s at 2500 rpm in a speed mixer.

### EXAMPLE 4

Zirconium dioxide powder ZrO₂ TZ-PX-245 from Tosoh yellow (B1a), ZrO₂ TZ-PX-364 from Tosoh white (B1b):

Preparation of the starting slips B1a and B1b: grinding 110 g of ZrO₂ powder with 1.0 g of anionic polyelectrolyte and 60 g of distilled water with 200 g ZrO₂ milling balls for 4 h, then sieving off the milling balls.

Preparing the slip for film casting: 114 g of starting slip B1a or B1b, 4.73 g of ethylene/vinyl acetate dispersion, 1.3 g of modified starch, 13.3 g of ethanol/2% MEK (MEK=methyl ethyl ketone), and 0.1 g of 1-octanol are stirred with 500 rpm for 1 h, then mixed for 20 s at 2500 rpm in a speed mixer.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

1. A molded part having a plurality of ceramic layers each formed from a ceramic material and provided on top of one another, wherein at least 21 layers are provided on top of one another.

2. The molded part according to claim 1, wherein each of the plurality of ceramic layers have has a thickness of from 1 μm to 120 μm.

3. The molded part according to claim 1, wherein the plurality of ceramic layers comprise the same ceramic material.

4. The molded part according to claim 1, wherein the ceramic material is selected from the group consisting of feldspar ceramics, metal oxide ceramics, non-oxidic ceramics, glass ceramics, and a combination thereof.

5. The molded part according to claim 1, wherein at least one ceramic layer has a porosity that is different from a porosity of the other ceramic layers.

6. The molded part according to claim 1, wherein the plurality of ceramic layers consist of different ceramic materials or ceramic materials having different physical properties.

7. The molded part according to claim 1, wherein the ceramic material of at least one of the plurality of ceramic layers includes at least one aggregate.

8. The molded part according to claim 7, wherein said aggregate is selected from the group consisting of dyeing substances, nucleating agents, fluxes, refining agents, ceramic fibers, nanomaterials, stabilizers, and a mixture thereof.

9. A process for preparing a molded part according to claim 1, comprising the following steps:
a) providing a slip comprising the ceramic material;
b) applying the slip to a support material in a desired layer thickness to form a layer;
c) drying the layer from step b) to obtain a single ceramic layer;
d) repeating steps a) to c) until the desired number of ceramic layers is achieved,
e) arranging the desired number of ceramic layers from step d) to obtain a stack arrangement; and
f) pressing the stack arrangement from step e) to obtain a molded part.

10. A dental restoration comprising the molded part according to claim 1.

11. The dental restoration according to claim 10, wherein said dental restoration product includes artificial teeth, an inlay, an only, a bride, or a crown.

12. (canceled)

13. A process for preparing a dental restoration, the process comprising:
shaping the molded part according to claim 1 by machine processing.

14. The molded part according to claim 1, wherein at least one ceramic layer comprises a different ceramic material compared to the ceramic material of the other ceramic layers.

15. The molded part according to claim 1, wherein each of the plurality of ceramic layers has a thickness of from 1 to 99 μm.

16. The molded part according to claim 1, wherein each of the plurality of ceramic layers has a thickness of from 10 μm to 80 μm.

17. The molded part according to claim 1, wherein each of the plurality of ceramic layers has a thickness of from 30 μm to 60 μm.

18. The dental restoration according to claim 10, wherein preparation of said dental restoration is effected by a computer-aided method.
